(19) United States
(12) Patent Application Publication
(10) Pub. No.: US 2009/0264888 A1
(43) Pub. Date: Oct. 22, 2009

(54) Bone graft drill

(75) Inventors: Stefan Neumeyer, Eschlkam (DE); Martin Teller, Lemgo (DE); Michael Kuellmer, Lemgo (DE)

Correspondence Address: KOLISCH HARTWELL, P.C., 200 PACIFIC BUILDING, 520 SW YAMHILL STREET, PORTLAND, OR, 97204, US

(73) Assignee: Gebr. Brasseler GmbH & Co. KG, Lemgo (DE)

(21) Appl. No.: 12/386,477

(22) Filed: Apr. 16, 2009

(30) Foreign Application Priority Data
Apr. 22, 2008 (DE) 10 2008 020 178.2

(51) Int. Cl. A61B 17/56

(52) U.S. Cl. 606/80

(57) ABSTRACT

A bone graft drill includes a shaft with a shaft axis, and a head with a rear side connected to the shaft and being shaped substantially conical at a front portion. A guiding tip is formed at a front end of the head, and a chipping recess is also formed in the head with an axis being inclined diagonally with respect to the shaft axis. The chipping recess has an edge constituting at least one cutting edge and an opening toward the rear side of the head.

### CROSS -REFERENCE TO RELATED APPLICATION

This application claims priority to German Patent Application No. 10 2008 020 178.2, filed Apr. 22, 2008, which is hereby incorporated by reference.

### BACKGROUND

The present disclosure relates to a bone graft drill and in detail to a dental drill for obtaining a bone graft. During the implant treatment of the jaws of a patient, bone management requires generating a bone graft. The bone graft should be as large as possible, i.e. be available in the form of coarse chips. These coarse chips are then inserted into the portions to be structured by the dentist or surgeon, to build up bone tissue, where in turn the implants can be anchored.

### SUMMARY

It is an object underlying the present disclosure to provide a bone graft drill that is suited to produce coarse bone graft chips while having a simple structure and being manufactured at low costs. According to the present disclosure, a bone graft drill is provided which has a relatively thick head. This head is not provided with directly cutting edges, as is usual. Rather, the head is formed with or penetrated by a chipping recess from its front end portion to its rear end portion. A plurality of chipping recesses may also be provided according to the present disclosure. The chipping recess extends diagonally (i.e. penetrating the head transversely) and is inclined with respect to the shaft axis. The intersection curve or cut edge between the chipping recess and the contour of the head generates a cutting edge with which coarse bone chips can be cut or fined down. These chips are also long, so that they can be gripped and handled well by the surgeon. The generated chips are therefore excellently suited for a reconstruction of bone portions. Moreover, the small surface support due to the multi-sidedness of the drill's tip may result in friction heat being reduced and the bone not being thermally damaged. Also the cutting properties of the drill are positively influenced, such that a possible locking of the drill is prevented.

### BRIEF DESCRIPTION OF THE DRAWINGS

FIG. 1 shows a schematic side view of a first embodiment of a bone graft drill.

FIG. 2 shows a side view, analogous to FIG. 1, in a perspective rotated by 90°.

FIG. 3 shows an enlarged illustration of a head portion of the bone graft drill according to FIG. 1.

FIG. 4 shows an enlarged illustration of the head portion according to FIG. 2.

FIG. 5 shows a front view of the embodiment of FIGS. 1 to 4.

FIG. 6 shows a perspective view of another embodiment of a bone graft drill.

FIG. 7 shows a bottom view of the embodiment shown in FIG. 6.

FIG. 8 shows a perspective view of a bone graft drill without a collecting sleeve.

FIG. 9 shows a view, analogous to FIG. 8, including a collecting sleeve attached to the bone graft drill.

FIG. 10 shows a side view of the embodiment according to FIG. 9.

FIGS. 11-13 show partial sectional views, analogous to FIG. 10, of different positions of the collecting sleeve 9.

### DETAILED DESCRIPTION

In the embodiments, identical components are designated with identical reference numerals.

The bone graft drill according to the present disclosure includes a head 1 as well as an integrally connected shaft 2. The shaft 2 is, at its rear portion, provided with the usual clamping system for inserting the bone graft drill into a driving unit, such as an angular piece.

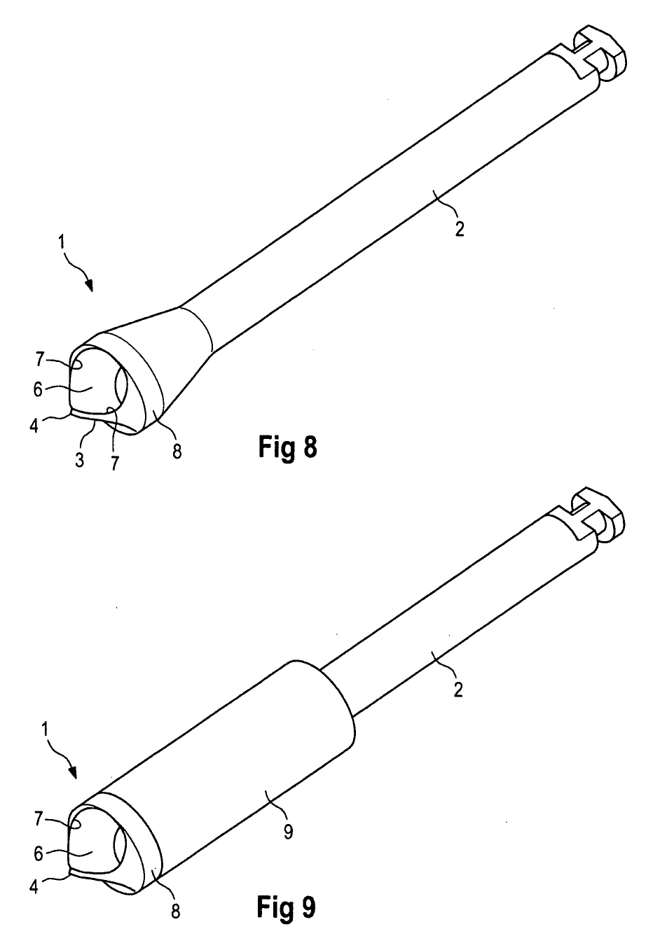

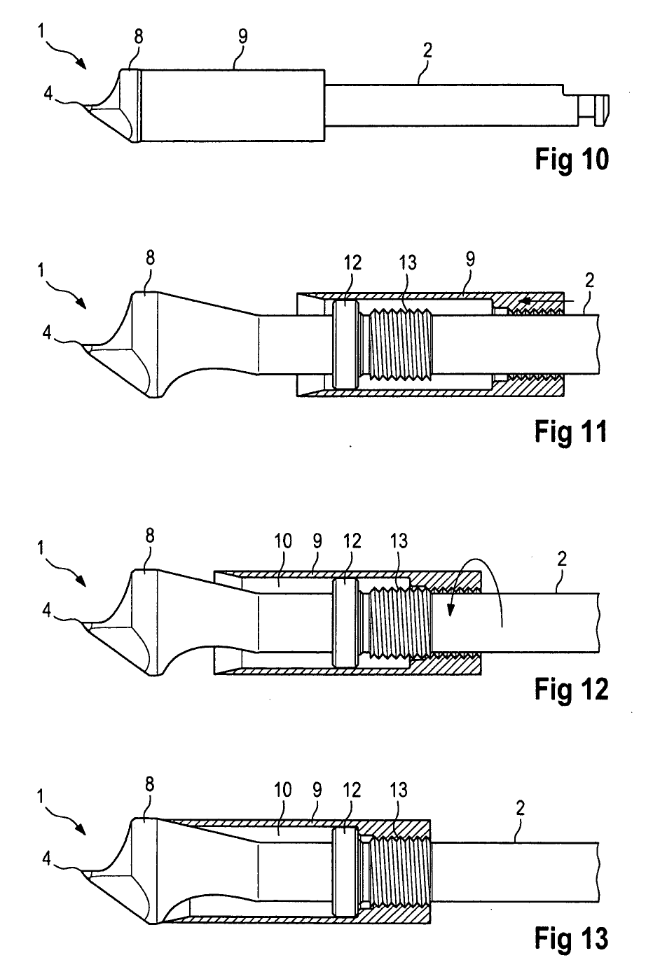

The head 1 includes a front portion 3 which is substantially conical and which is formed with a round cross-section in cutting planes perpendicular with respect to a shaft axis 5, as is for example shown in FIGS. 8 and 9.

According to the present disclosure, the head may also be provided with a front portion that is round shaped, i.e. having circular cross-sections in a vertical direction with respect to the rotary axis of the bone graft drill. Alternatively, the head may have a multi-sided contour or structure. The conical portion 3, for example, may also be provided with a multi-sided contour (see FIGS. 6 and 7).

The conical portion 3 has a guiding tip 4 at its front end, said tip being for example shaped like a needle and fixing the bone graft drill during operation. The needle-shaped guiding tip 4 serves to center the bone graft drill. This tip 4 is, in addition, self-centering, formed symmetrically and teethed.

The head 1 is further provided with a cylindrical wall portion 8 which is adjacent the conical portion 3. The portion of the head 1 facing the shaft 2 is formed conically tapered and merges evenly into the shaft 2.

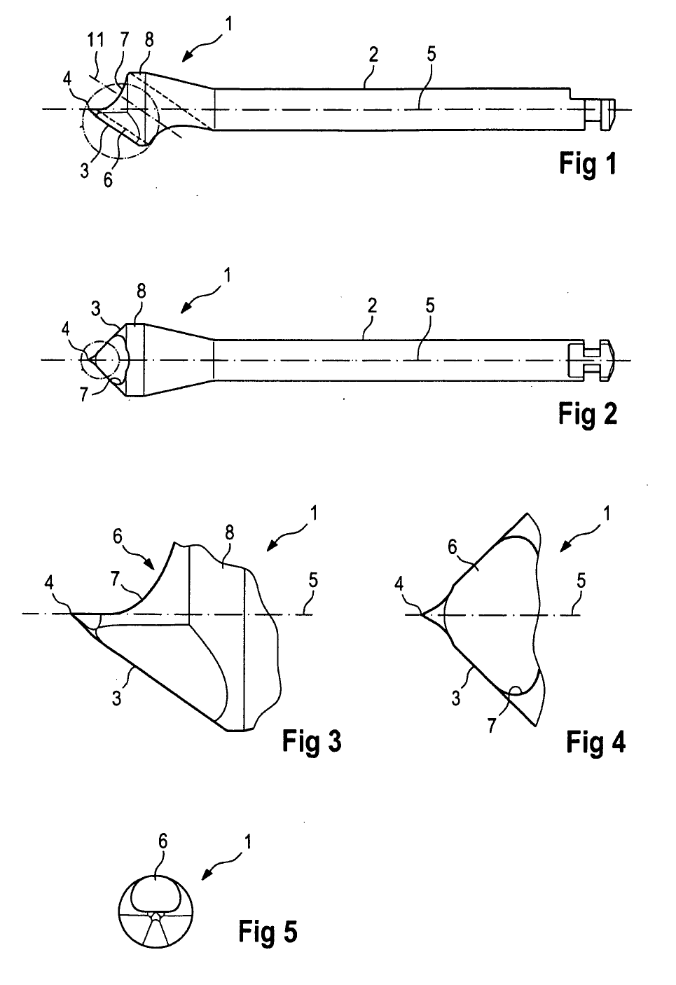

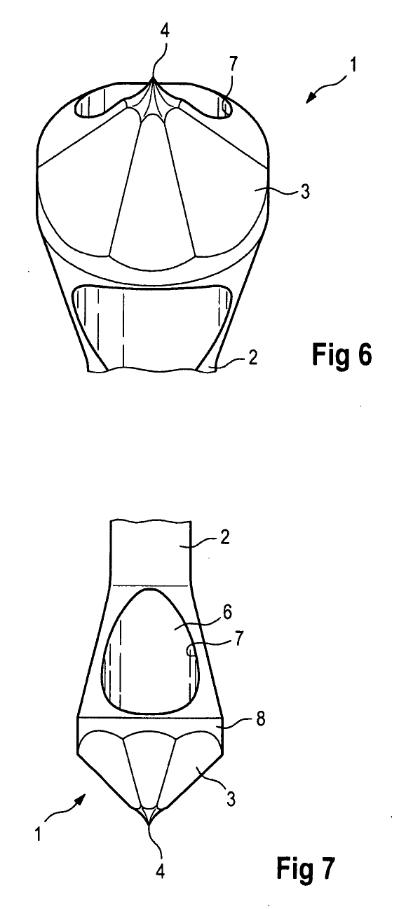

According to the present disclosure, a substantially cylindrical chipping recess 6 penetrates the head 1, wherein an axis 11 of the recess is inclined in an angle with respect to the shaft axis 5, for example by 35°. The axis 11 is particularly shown in FIG. 1. Here, it is discernible that the chipping recess 6 extends from the conical portion 3 to the rear portion of the head 1.

According to the present disclosure, the center axis of the chipping recess 6 may also be inclined at an angle of 30° to 40° with respect to the shaft axis 5. The conical portion 3 is provided with a cone angle of substantially 90°, however, also smaller or larger conical angles are suitable according to the present disclosure.

The chipping recess 6 and the conical portion 3 of the head 1 together form an intersection edge 7, as it is shown in detail in FIGS. 1 and 2. This edge 7 is a cut edge by which chips are generated in the region of the front part of the head 1 when the bone graft drill is advanced.

According to the present disclosure, the chipping recess 6 is shaped cylindrically, thereby to simplify the transport of the chips. It is also possible to expand the chipping recess 6 slightly toward the rear portion of the head 1, i.e. toward the shaft 2, in order to avoid clogging of the chipping recess 6 with chips in this way.

The relatively coarse chips are fed through the head 1 toward the rear end portion of the chipping recess 6. At this point, they are either extracted or collected in a chipping space 10 of a chipping sleeve 9.

According to the present disclosure, the head 1 has a larger diameter than the shaft 2 of the bone graft drill. In this way, removal of the chips toward the shaft 2 is possible.

The head 1 comprises a cylindrical wall portion adjacent the conical portion 3 that serves as a guide during use of the bone graft drill, and may co-operate with a collecting sleeve 9 which is supported axially displaceably on the shaft 2 in a further development of the present disclosure. Herein, the collecting sleeve 9 forms a space in which the chips can be collected. The collecting sleeve 9 can be axially fixed, for example by way of a threaded screwing.

As is shown in FIGS. 11 to 13, the collecting sleeve 9 is supported on the shaft 2 to be axially displaceable and forms a chipping room 10 which is delimited toward its front end portion by the head 1 and toward its rear end portion by an annular bead 12, when the collecting sleeve 9 is in an operating position (see FIG. 13). The collecting sleeve 9 is provided with an internal thread which can be screwed on a thread 13 of the shaft 2, as it is shown in FIGS. 11 to 13.

The chips generated by the inventive bone graft drill can be collected by the collecting sleeve 9 and the space formed thereby, as described above. However, it is also possible to suction the chips with a saliva suction device, and collect them in a bone filter located near the saliva suction device.

Further, the inventive bone graft drill can be used with a slow rotational speed in the range of 250 to 1000 rpm. Therewith, a low-vibration and centered operation of the bone graft drill is secured.

Due to its symmetric structure, the inventive bone graft drill may be used clockwise or counter-clockwise.

We claim:

1. A bone graft drill comprising:
a shaft including a shaft axis,
a head including a rear side connected to the shaft and being shaped substantially conical at a front portion and including a guiding tip at a front end, and a chipping recess penetrating the head and having an axis being inclined diagonally with respect to the shaft axis, wherein the chipping recess has an edge constituting at least one cutting edge and an opening toward the rear side of the head.

2. The bone graft drill of claim 1, wherein the substantially conical front portion of the head is round.

3. The bone graft drill of claim 1, wherein the substantially conical front portion of the head is multi-sided.

4. The bone graft drill as in any one of claims claims 1, 2, and 3, wherein the chipping recess is shaped substantially cylindrically.

5. The bone graft drill of claim 1, wherein the head has a diameter larger than a diameter of the shaft.

6. The bone graft drill of claim 1, wherein the head further includes a cylindrical wall portion adjacent to the substantially conical front portion.

7. The bone graft drill of claim 1, wherein the chipping recess is inclined with respect to the shaft axis by an angle between about 30° and about 40°.

8. The bone graft drill of claim 7, wherein the substantially conical front portion has a cone angle of about 90°.

9. The bone graft drill of claim 1, wherein a collecting sleeve is supported axially displaceably on the shaft, and wherein the collecting sleeve forms a chipping space.

10. The bone graft drill of claim 9, wherein the collecting sleeve is axially fixable.

11. The bone graft drill of claim 10, wherein the collecting sleeve is screwable to the shaft by a thread to be axially fixable.

12. A bone graft drill comprising:
a shaft including a shaft axis,
a head including a rear side connected to the shaft and being shaped substantially conical at a front portion and including a guiding tip at a front end, and
a substantially cylindrical chipping recess penetrating the head and having an axis being inclined with respect to the shaft axis, wherein the chipping recess has at least one cutting edge.

13. The bone graft drill of claim 12, wherein the chipping recess is inclined with respect to the shaft axis by an angle between about 30° and about 40°.

14. The bone graft drill of claim 12, wherein the substantially conical front portion has a cone angle of about 90°.

15. The bone graft drill of claim 12, wherein the guiding tip is needle-shaped.

16. A bone graft drill comprising:
a shaft including a shaft axis,
a head including a rear side connected to the shaft at a front portion and including a guiding tip at a front end,
a chipping recess penetrating the head and having an axis being inclined with respect to the shaft axis, wherein the chipping recess has an edge constituting at least one cutting edge and an opening toward the rear side of the head, and
a collecting sleeve coupled axially displaceably on the shaft, wherein the collecting sleeve forms a chipping space.

17. The bone graft drill of claim 16, further wherein the collecting sleeve is axially fixable.

18. The bone graft drill of claim 16, further wherein the collecting sleeve is screwable to the shaft by a thread to be axially fixed.
